(19) United States
(12) Patent Application Publication
(10) Pub. No.: US 2014/0206617 A1
(43) Pub. Date: Jul. 24, 2014

(54) USE OF ANTITHROMBIN IN THE TREATMENT OF PRE-ECLAMPSIA

(71) Applicant: rEVO Biologics, Inc., Framingham, MA (US)

(72) Inventors: Johan Frieling, Natick, MA (US); Simon Lowry, Hillsdale, NY (US)

(73) Assignee: rEVO Biologics, Inc., Framingham, MA (US)

(21) Appl. No.: 14/239,869

(22) PCT Filed: Mar. 12, 2013
(86) PCT No.: PCT/US13/30350

§ 371 Date: Feb. 20, 2014

Related U.S. Application Data
(60) Provisional application No. 61/609,534, filed on Mar. 12, 2012.

(51) Int. Cl. A61K 38/57

(52) U.S. Cl. CPC A61K 38/57
USPC 514/14.7

(57) ABSTRACT

In one aspect, the disclosure provides methods for the treatment of pre-eclampsia and severe pre-eclampsia comprising administering antithrombin. In some embodiments, the antithrombin used in the methods disclosed herein is ATryn®.

### RELATED APPLICATIONS

This application claims the benefit under 35 U.S.C. §119 of U.S. provisional application 61/609,534 filed Mar. 12, 2012, the entire contents of which is incorporated herein by reference.

### FIELD OF THE INVENTION

The disclosure relates to the treatment of pre-eclampsia and severe pre-eclampsia.

### BACKGROUND OF THE INVENTION

Pre-eclampsia and severe pre-eclampsia are conditions that can occur during pregnancy including during the pre-term and near term stages of pregnancy. The conditions are characterized by hypertension and increased amounts of protein in the urine. If not properly treated, pre-eclampsia and severe pre-eclampsia can lead to seizures and even death. New treatment methods for pre-eclampsia and severe pre-eclampsia are needed therefore.

### SUMMARY OF THE INVENTION

In one aspect, the disclosure provides methods of treating pre-eclampsia and severe pre-eclampsia.

In one aspect, the disclosure provides a method of treating pre-eclampsia in a subject comprising administering to a subject having pre-eclampsia a therapeutically effective amount of antithrombin to treat the pre-eclampsia. In some embodiments, the pre-eclampsia is severe pre-eclampsia. In some embodiments, the subject is at less than 24 weeks of pregnancy. In some embodiments, the subject is at between 24 weeks and 28 weeks of pregnancy. In some embodiments, the subject is at between 28 weeks and 32 weeks of pregnancy. In some embodiments, the subject is at between 28 weeks and 34 weeks of pregnancy. In some embodiments, the subject is at over 34 weeks of pregnancy.

In some of the embodiments of the methods provided herein, the antithrombin has a high mannose glycosylation pattern. In some embodiments, the antithrombin comprises GalNac (N-acetylgalactosamine). In some embodiments, the antithrombin has a fucose-GlcNAc glycosylation pattern. In some embodiments, the antithrombin is transgenically produced antithrombin. In some embodiments, the antithrombin is transgenically produced in a goat. In some embodiments, the antithrombin is ATryn®.

In some of the embodiments of the methods provided herein, the antithrombin is administered at a dose of 1500 units per day. In some embodiments, the antithrombin is administered at a dose of 3000 units per day. In some embodiments, the antithrombin is administered at a dose of 12,000 units per day. In some embodiments, the antithrombin is administered through continuous infusion. In some embodiments, the antithrombin is administered as a bolus. In some embodiments, the antithrombin is administered at a dose of 3500 units twice daily by bolus infusion. In some embodiments, the antithrombin is administered at a dose of 10,500 units daily by continuous infusion. In some embodiments, the antithrombin is administered at a dose of 12,000 units daily by continuous infusion.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the Figures. The invention is capable of other embodiments and of being practiced or of being carried out in various ways. Also, the phraseology and terminology used herein is for the purpose of description and should not be regarded as limiting.

### BRIEF DESCRIPTION OF THE DRAWINGS

N/A

### DETAILED DESCRIPTION OF THE INVENTION

In one aspect, the disclosure provides methods of treating pre-eclampsia. In some embodiments, the method of treating pre-eclampsia comprises administering to a subject having pre-eclampsia a therapeutically effective amount of antithrombin to treat the pre-eclampsia. In some embodiments, the pre-eclampsia is severe pre-eclampsia. In some embodiments, the antithrombin used in the methods disclosed herein is ATryn®.

### Pre-Eclampsia and Severe Pre-Eclampsia

Pre-eclampsia is a condition characterized by hypertension and increased amounts of protein in the urine. Pre-eclampsia can occur during pregnancy and is thought to affect around 6-8% of all pregnancies. Pre-eclampsia is diagnosed by evaluating the combination of hypertension (equal to or over 140/90 mm Hg of systolic/diastolic blood pressure) and proteinuria (at least 300 mg of protein in a 24 hour urine sample). Severe pre-eclampsia is generally characterized by a blood pressure of at least 160/110 mm Hg of systolic/diastolic blood pressure and can occur with or without other symptoms.

Onset of pre-eclampsia generally occurs after the 32^{nd} week of pregnancy. However, pre-eclampsia may develop as early as 20 weeks of pregnancy, and early onset of pre-eclampsia has been associated with increased morbidity. Pre-eclampsia and severe pre-eclampsia may occur during the severe preterm (generally defined as prior to the 28^{th} week of pregnancy), preterm (generally defined as between the 28^{th} and 34^{th} week of pregnancy) and near term, also called late preterm (generally defined as between the 34^{th} and 37^{th} week of pregnancy).

The causes of pre-eclampsia and severe pre-eclampsia are not well understood. If not properly treated, pre-eclampsia can lead to eclampsia which, in turn, can result in seizures and even death.

### Treatment of Pre-Eclampsia and Severe Pre-Eclampsia

Traditional treatment of pre-eclampsia and eclampsia includes antihypertensive therapy to lower hypertension and the infusion of magnesium sulfate to prevent seizures. However, delivery is the only real cure for pre-eclampsia.

Treatment of pre-eclampsia and severe pre-eclampsia, as used herein, refers to the improvement in any of the physiological parameters associated with the condition of pre-eclampsia and severe pre-eclampsia. Thus, for instance, treatment in pre-eclampsia includes a lowering of the blood pressure and suppression of other manifestations such as the chance of seizure. Physiological parameters that reflect treatment of pre-eclampsia include the lowering of the level of fibronectin, C-reactive protein, elastase and tissue plasminogen activator levels (See e.g., Paternoster et al. Thromb. Haemost. 2004, 91: 283-289).

It has been found that antithrombin can be used in the treatment of pre-eclampsia. In addition, certain forms of antithrombin, such as ATryn®, can exhibit different properties that can affect treatment of pre-eclampsia and severe pre-eclampsia.

### Antithrombin and ATryn®

In one aspect, the disclosure provides methods for the treatment of pre-eclampsia and severe pre-eclampsia comprising administering antithrombin. In some embodiments, the antithrombin has a high mannose glycosylation pattern. In some embodiments, the antithrombin comprises GalNac (N-acetylgalactosamine). In some embodiments, the antithrombin has a fucose-GlcNAc glycosylation pattern. In some embodiments, the antithrombin is transgenically produced antithrombin, such as in the mammary gland. In some embodiments, the antithrombin is transgenically produced in a goat. In some embodiments, the antithrombin is ATryn® (See e.g., U.S. Pat. No. 5,843,705, U.S. Pat. No. 6,441,145, U.S. Pat. No. 7,019,193 and U.S. Pat. No. 7,928,064, which are all incorporated by reference in their entirety).

Antithrombin is a glycoprotein of about 58 kDA. Antithrombin is a serine protease inhibitor that inhibits thrombin and Factor Xa. Antithrombin is naturally found in the serum of mammals including humans. The physiological level of antithrombin in human serum from a healthy individual is about 14-20 mg/dL.

Traditionally, the term “antithrombin” relates to a family of closely related proteins that includes antithrombin I, antithrombin II, antithrombin III and antithrombin IV. However, antithrombin III is the only member of the antithrombin family that has been associated with a significant physiological function and the current literature often uses the terms antithrombin and antithrombin III interchangeably. Antithrombin, as used herein, refers to antithrombin III and any antithrombin that has the same, or a similar, activity as antithrombin III.

Antithrombin is a glycoprotein and the human antithrombin includes four glycosylation sites: Asn96, Asn135, Asn155 and Asn192. Antithrombin occurs both in an alpha form (alpha-antithrombin) and in a beta form (beta-antithrombin), with the alpha form being the most prevalent. The beta form of human antithrombin can be distinguished from the alpha form of human antithrombin because the beta form is not glycosylated at Asn135. In some embodiments, antithrombin, as used in the methods disclosed herein, includes both the major alpha form of antithrombin and the minor beta form of antithrombin. In some embodiments, antithrombin, as used in the methods disclosed herein, is alpha-antithrombin.

Antithrombin is conserved between mammalian species with only minor differences in amino acid sequence. The human antithrombin III is 432 amino acids in length. Non-human human antithrombins are the same length or are similar in length (e.g., 433 amino acids). In some embodiments, the species of antithrombin used in the treatment of a subject according to the methods disclosed herein is the same species as the subject. Thus, for instance, human antithrombin is used in methods of treatment in humans, while bovine antithrombin is used in methods of treatment in bovines.

It should further be appreciated that in addition to the amino acid sequence the glycosylation of antithrombin is also species-specific. Thus, for instance, human antithrombin isolated from human plasma (plasma-derived human antithrombin), has a different glycosylation pattern than goat antithrombin isolated from goat plasma. However, as explained below, human antithrombin, for example, may be produced in a goat, providing human antithrombin (i.e., antithrombin with a human amino acid sequence) with a glycosylation pattern that mimics the glycosylation of goat antithrombin.

In some embodiments, the antithrombin used in the methods disclosed herein has a high mannose glycosylation pattern. Antithrombin with a high mannose glycosylation pattern, as used herein, refers to an antithrombin in which one or more of the glycosylation side chains predominantly comprise an oligomannose or a hybrid type oligosaccharide (In contrast to side chains comprising bi-antennary complex oligosaccharides, which are the predominant side-chain structure found in plasma-derived human antithrombin). In some embodiments, the antithrombin used in the methods disclosed herein comprises GalNac (N-acetylgalactosamine). In some embodiments, the antithrombin used in the methods disclosed herein has a fucose-GlcNAc glycosylation pattern. A fucose-GlcNAc glycosylation pattern, as used herein, refers to antithrombin that has fucose on its proximal GlcNac on a majority of the glycosylation sites that have complex oligosaccharides. In some embodiments, the antithrombin used in the methods disclosed herein has a high mannose pattern, includes GalNac and includes a fucose-GlcNAc glycosylation pattern. It should be noted that human antithrombin (i.e., antithrombin with a human amino acid sequence) that is transgenically produced in goats has a high mannose pattern, includes GalNac and includes a fucose-GlcNAc glycosylation pattern, while plasma-derived human antithrombin does not have these glycosylation patterns (See e.g., U.S. Pat. No. 5,843,705, U.S. Pat. No. 6,441,145, U.S. Pat. No. 7,019,193, and U.S. Pat. No. 7,928,064).

The antithrombin used in the methods disclosed herein can be produced through a variety of methods. In some embodiments, the antithrombin is produced by isolation from plasma (i.e., plasma-derived antithrombin). In some embodiments, the antithrombin is recombinantly produced. In some embodiments, the antithrombin is transgenically produced (See e.g., U.S. Pat. No. 5,843,705, U.S. Pat. No. 6,441,145, U.S. Pat. No. 7,019,193, U.S. Pat. No. 7,928,064, U.S. Pat. No. 6,268,487, U.S. Pat. No. 7,045,676, U.S. Pat. No. 7,939,317, and U.S. Pat. No. 7,521,632, which are all incorporated by reference in their entirety).

In some embodiments, the antithrombin used in the methods disclosed herein is transgenically produced. In some embodiments, the transgenically produced antithrombin is produced in a mammal. In some embodiments, the transgenically produced antithrombin is produced in an ungulate. In some embodiments, the transgenically produced antithrombin is produced in a goat. It should be appreciated that the antithrombin produced in a first species can be an antithrombin from a second species. Thus, for instance, human antithrombin can be transgenically produced in mice and goats. Similarly, bovine antithrombin can also be transgenically produced in mice and goats. In addition, antithrombin can also be transgenically produced in the species of origin. Thus, goat antithrombin can be transgenically produced in goats.

In some embodiments, the antithrombin used in the methods disclosed herein is transgenically produced. In some embodiments, the transgenically produced antithrombin has a glycosylation pattern that is different from plasma-derived antithrombin. In general, the glycosylation pattern of the antithrombin depends on the species of animal the antithrombin is produced in. Thus, for instance, antithrombin transgenically produced in mice is expected to have a different glycosylation pattern than antithrombin produced in goats.

In some embodiments, the antithrombin used in the methods disclosed herein has the glycosylation pattern of antithrombin transgenically produced in goats.

It should be appreciated that the glycosylation pattern of transgenically produced antithrombin will also depend on the nature of the organ, or body part, of the transgenic animal in which the protein is produced. Thus, the glycosylation pattern of antithrombin produced in the mammary gland is expected to be different from antithrombin produced in the blood, even if produced in the same species. In some embodiments, the antithrombin used in the methods disclosed herein is produced in the mammary gland of goats.

It should further be appreciated that antithrombin with the glycosylation pattern of antithrombin produced in goats can also be provided by producing the antithrombin in a species other than the goat and modifying the glycosylation pattern in downstream processing. For instance, glycosylated antithrombin may be produced in mice and the glycosylation pattern of the mice-produced antithrombin may be altered to generate the glycosylation pattern of goat antithrombin by in vitro modification. For instance, the mice-produced antithrombin may be altered through the action of glycosylases or transferases. In addition, the glycosylation pattern may be modified by non-enzymatic (i.e., synthetic) methods.

Antithrombin with the glycosylation pattern of goat produced antithrombin may also be provided by producing antithrombin in cells (e.g., insect cells, bacterial cells) and adding or modifying the glycosylation pattern in downstream processing. Alternatively, antithrombin with the glycosylation pattern of goat produced antithrombin may be provided by isolation from plasma from a non-goat species and the glycosylation pattern may subsequently be modified in downstream processing.

In some embodiments, the antithrombin used in the methods disclosed herein is ATryn®. ATryn® is a transgenic human alpha antithrombin that is produced in the goat mammary gland (See e.g., U.S. Pat. No. 5,843,705, U.S. Pat. No. 6,441,145, U.S. Pat. No. 7,019,193, and U.S. Pat. No. 7,928,064). ATryn® is approved by the FDA for the prevention of peri-operative and peri-partum thromboembolic events in hereditary antithrombin deficient patients. In Europe, ATryn® is approved for use in surgical patients with congenital antithrombin deficiency for the prophylaxis of deep vein thrombosis and thromboembolism in clinical risk situations.

The glycosylation pattern of the goat-produced (human) antithrombin ATryn® differs from the glycosylation pattern of plasma-derived human antithrombin. Because the glycosylation pattern is different, ATryn® shows biochemical and physiological properties that are different from plasma-derived human antithrombin. For instance, ATryn® shows a fourfold higher affinity for heparin than plasma-derived antithrombin (Edmunds et al., Blood, 1998, 91: 4561-4571). In some embodiments, ATryn® binds heparin sulfate receptors with a higher affinity than plasma-derived antithrombin. When the biodistribution of plasma-derived antithrombin and ATryn® were compared, circulating ATryn® was more rapidly removed from circulation by binding to blood vessel walls and distribution to the liver (see e.g., Berry et al., Thromb. Haemost. 2009, 102: 302-308).

### Subject

In one aspect, the disclosure provides methods for the treatment of pre-eclampsia and severe pre-eclampsia in a subject. A “subject”, as used herein, is a human or other vertebrate mammal including, but not limited to, mouse, rat, dog, cat, horse, cow, pig, sheep, goat, or non-human primate.

Pre-eclampsia and severe pre-eclampsia are associated with pregnancy and a subject therefore refers to a female. In some embodiments, the female is in a severe preterm, preterm or near term stage of pregnancy.

In some embodiments, the subject is a human female, i.e., a woman. In some embodiments, the woman is in severe preterm pregnancy, preterm pregnancy, or near term pregnancy, also called late preterm pregnancy. In some embodiments, the subject is at less than 24 weeks of pregnancy. In some embodiments, the subject is at between 24 weeks and 28 weeks of pregnancy. In some embodiments, the subject is at between 28 weeks and 32 weeks of pregnancy. In some embodiments, the subject is at between 28 weeks and 34 weeks of pregnancy. In some embodiments, the subject is at over 34 weeks of pregnancy.

### Additional Therapies

In some embodiments, the methods of the treatment of pre-eclampsia and severe pre-eclampsia comprising the administration of antithrombin are coupled with additional therapies. In some embodiments, additional therapies are therapies that are used to treat pre-eclampsia and severe pre-eclampsia or pathologies associated with pre-eclampsia and severe pre-eclampsia. Additional therapies include the administration of anti-hypertensive drugs and the administration of magnesium sulfate.

In some embodiments, additional therapies are therapies generally associated with promoting the well-being of mother and fetus during pregnancy. In some embodiments, the additional therapy includes the administration of analgesics. In some embodiments, the additional therapy includes the administration of corticosteroids, e.g., to promote fetal lung development. A medical professional will know what therapies and drugs can be safely administered during pregnancy.

### Therapeutically Effective Amount

In one aspect, the disclosure provides methods for the treatment of pre-eclampsia and severe pre-eclampsia comprising administering antithrombin. In some embodiments, antithrombin is administered in therapeutically effective amounts to treat pre-eclampsia and severe pre-eclampsia. The terms “therapeutically effective amount” and “effective amount”, which are used interchangeably, refer to the amount necessary or sufficient to realize a desired therapeutic effect, e.g., the treatment of pre-eclampsia and severe pre-eclampsia. Combined with the teachings provided herein, by choosing among the various active compounds and weighing factors such as potency, relative bioavailability, subject body weight, severity of adverse side-effects and preferred mode of administration, an effective prophylactic or therapeutic treatment regimen can be selected which does not cause substantial toxicity and yet is effective to treat the particular subject.

The effective amount for any particular application can vary depending on such factors as the disease or condition being treated, the particular therapeutic agent(s) to be administered, the size of the subject, or the severity of the disease or disorder. One of ordinary skill in the art can empirically determine the effective amount of antithrombin without necessitating undue experimentation. It is preferred generally that a maximum dose be used, that is, the highest safe dose according to some medical judgment. Multiple doses per day, week or month may be contemplated to achieve appropriate systemic levels of antithrombin. Appropriate system levels can be determined by, for example, measurement of the patient's peak or sustained plasma level of antithrombin.

Doses of antithrombin (e.g., ATryn®) to be administered are generally expressed in mg/kg, units of antithrombin per kg or units of antithrombin per day. In some embodiments, antithrombin is administered at a dose of 10 units per day or more, 50 units per day or more, 100 units per day or more, 200 units per day or more, 500 units per day or more, 1000 units per day or more, 1500 units per day or more, 2000 units per day or more, 2500 units per day or more, 3000 units per day or more, 3500 units per day or more, 4000 units per day or more, 5000 units per day or more, 4500 units per day or more, 5000 units per day or more, 5500 units per day or more, 6000 units per day or more, 6500 units per day or more, 7000 units per day or more, 7500 units per day or more, 8000 units per day or more, 8500 units per day or more, 9000 units per day or more, 9500 units per day or more, 10,000 units per day or more, 10,500 units per day or more, 11,000 units per day or more, 11,500 units per day or more, 12,000 units per day or more, 12,500 units per day or more, 13,000 units per day or more, 13,500 units per day or more, 14,000 units per day or more, 14,500 units per day or more, 15,000 units per day or more, 16,000 units per day or more, 17,000 units per day or more, 18,000 units per day or more, 19,000 units per day or more, or 20,000 units per day or more. In some embodiments, the antithrombin is administered at a dose of 1500 units per day. In some embodiments, the antithrombin is administered at a dose of 3000 units per day. In some embodiments, the antithrombin is administered at a dose of 7000 units per day. In some embodiments, the antithrombin is administered at a dose of 10,500 units per day. In some embodiments, the antithrombin is administered at a dose of 12,000 units per day.

In some embodiments, the antithrombin is administered at two doses of 3500 units twice daily. In some embodiments, the antithrombin is administered at two doses of 3500 units twice daily by bolus infusion (IV injection). In some embodiments, the antithrombin is administered at a dose of 10,500 units per day. In some embodiments, the antithrombin is administered at a dose of 10,500 units per day by continuous infusion. In some embodiments, the antithrombin is administered at a dose of 12,000 units per day. In some embodiments, the antithrombin is administered at a dose of 12,000 units per day by continuous infusion.

It should be appreciated that amounts of antithrombin can also be expressed in weight (e.g., mg) rather than units. Generally, 1 mg of antithrombin corresponds to 6-7 units of antithrombin III, dependent on the lot, method of manufacturing, etc.

In some embodiments, antithrombin is administered at a dose of 1 unit per kg or more, 1 unit per kg or more, 5 units per kg day or more, 10 units per kg or more, 20 units per kg or more, 30 units per kg or more, 40 units per kg or more, 50 units per kg or more, 100 units per kg or more, 150 units per kg or more, 200 units per kg or more, 250 units per kg or more, 300 units per kg or more, 350 units per kg or more, 400 units per kg or more, 450 units per kg or more, 500 units per kg or more, 600 units per kg or more, 700 units per kg or more, 800 units per kg or more, 900 units per kg or more, or 1000 units per kg or more.

In some embodiments, the therapeutically effective amount is administered in one dose. In some embodiments the therapeutically effective amount is administered in multiple doses. Dosage may be adjusted appropriately to achieve desired levels of antithrombin, local or systemic, depending upon the mode of administration. In the event that the response in a subject is insufficient at such doses, even higher doses (or effective higher doses by a different, more localized delivery route) may be employed to the extent that subject tolerance permits. Multiple doses per day may be contemplated to achieve appropriate systemic levels of compounds.

### Administration

In one aspect, the disclosure provides methods for the treatment of pre-eclampsia and severe pre-eclampsia comprising administering antithrombin. In some embodiments, the antithrombin is administered through bolus infusion. In some embodiments, the antithrombin is administered through continuous infusion.

Antithrombin is typically administered to subjects as pharmaceutical compositions, which may routinely contain pharmaceutically acceptable concentrations of salt, buffering agents, preservatives, compatible carriers, adjuvants, and optionally other therapeutic ingredients. The nature of the pharmaceutical carrier and other components of the pharmaceutical composition will depend on the mode of administration.

The pharmaceutical compositions of the disclosure may be administered by any means and route known to the skilled artisan in carrying out the treatment methods described herein. Preferred routes of administration include but are not limited to oral, intravenous, subcutaneous, parenteral, intratumoral, intramuscular, intranasal, intracranial, sublingual, intratracheal, inhalation, ocular, vaginal, and rectal.

Antithrombin, when it is desirable to deliver systemically, may be formulated for parenteral administration by injection or infusion, e.g., by bolus injection, bolus IV injection, bolus infusion, continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

For oral administration, antithrombin can be formulated readily by combining the compounds with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl- cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally, the oral formulations may also be formulated in saline or buffers, e.g., EDTA for neutralizing internal acid conditions, or may be administered without any carriers.

For oral delivery, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Examples of the more common inert ingredients that are used as enteric coatings are cellulose acetate trimellitate (CAT), hydroxypropylmethyl-cellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films. A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic powder; for liquid forms, a soft gelatin shell may be used. The shell material of cachets could be thick starch or other edible paper. For pills, lozenges, molded tablets or tablet triturates, moist massing techniques can be used.

Antithrombin can be included in the formulation as fine multi-particulates in the form of granules or pellets. The formulation of the material for capsule administration could also be as a powder, lightly compressed plugs or even as tablets. The pharmaceutical composition could be prepared by compression. One may dilute or increase the volume of the pharmaceutical composition with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the pharmaceutical composition, such as in a solid dosage form. Materials used as disintegrants include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may also be used. Binders may be used to hold the therapeutic agent together to form a hard tablet and include materials from natural products such as acacia, tragacanth, starch and gelatin. An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Glidants that might improve the flow properties of the drug during formulation and to aid rearrangement during compression might be added. The glidants may include starch, talc, pyrogenic silica and hydrated silicoaluminate.

For administration by inhalation, antithrombin may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas.

Also contemplated herein is pulmonary delivery of antithrombin. Antithrombin may be delivered to the lungs of a mammal for local or systemic delivery. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (al-antitrypsin); Smith et al., 1989, J. Clin. Invest. 84:1145-1146 (a-l-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Nasal delivery of a pharmaceutical composition comprising antithrombin is also contemplated. Nasal delivery allows the passage of a pharmaceutical composition to the blood stream directly after administering the composition to the nose, without the necessity for deposition of the product in the lung.

In some embodiments antithrombin is administered locally. Local administration methods are known in the art and will depend on the target area or target organ. Local administration routes include the use of standard topical administration methods such as epicutaneous (application onto the skin), by inhalational, rectal (e.g., by enema or suppository), by eye drops (onto the conjunctiva), ear drops, intranasal route, and vaginal.

Antithrombin may also be formulated in rectal or vaginal compositions such as suppositories or retention enemas, e.g., containing conventional suppository bases such as cocoa butter or other glycerides. In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble analogs, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose analogs, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or one or more auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, 1990, Science 249, 1527-1533, which is incorporated herein by reference.

The agents and compositions described herein may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

The pharmaceutical compositions of the disclosure contain an effective amount of antithrombin included in a pharmaceutically-acceptable carrier. The term pharmaceutically acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compositions of the present disclosure, and with each other, in a manner such that there is no interaction which would substantially impair the desired pharmaceutical efficiency.

Both non-biodegradable and biodegradable polymeric materials can be used in the manufacture of particles for delivering the compositions of the disclosure. Such polymers may be natural or synthetic polymers. The polymer is selected based on the period of time over which release is desired. Bioadhesive polymers of particular interest include bioerodible hydrogels described by Sawhney et al., 1993, Macromolecules 26, 581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Antithrombin may be contained in controlled release systems. The term “controlled release” is intended to refer to any agents and compositions described herein-containing formulation in which the manner and profile of agents and compositions described herein release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a compound over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the compound therefrom. “Delayed release” may or may not involve gradual release of a compound over an extended period of time, and thus may or may not be “sustained release.” Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

### Kits

In one aspect, the disclosure provides kits comprising antithrombin (e.g., ATryn®). In some embodiments, the antithrombin is in sterile container(s). In some embodiments, the kit comprises a pharmaceutical carrier and instructions for administration of the kit components. In some embodiments, the kit includes a pharmaceutical preparation vial, a pharmaceutical preparation diluent vial, and the antithrombin. The diluent vial contains a diluent such as physiological saline for diluting what could be a concentrated solution or lyophilized powder of a composition of the disclosure. In some embodiments, the instructions include instructions for mixing a particular amount of the diluent with a particular amount of a concentrated pharmaceutical composition, whereby a final formulation for injection or infusion is prepared. In some embodiments, the instructions include instructions for use in a syringe or other administration device. In some embodiments, the instructions include instructions for treating a patient with an effective amount of a composition of the disclosure. It also will be understood that the containers containing the preparations, whether the container is a bottle, a vial with a septum, an ampoule with a septum, an infusion bag, and the like, may contain indicia such as conventional markings which change color when the preparation has been autoclaved or otherwise sterilized.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

### Example 1

### Treatment of Preterm Pre-Eclampsia with ATryn®

A multicenter, randomized, placebo-controlled, double-blind, phase 2 trial is performed to examine safety and efficacy of recombinant human antithrombin (ATryn®) in preterm pre-eclampsia (PPE). In the study, the safety of ATryn® in both mother and fetus/neonate is assessed, the pharmacokinetics of ATryn® in PPE in mother and neonate at delivery are assessed, and the efficacy of ATryn® for the treatment of PPE in addition to expectant management to prolong gestational age at delivery is assessed.

Methods: Sixty women aged ≧18 years-old who are at 24 to 28 weeks of gestation and who have hypertension and proteinuria (≧160/≧110 mm Hg and dipstick 1+ or protein/creatinine assessment; or <160/<110 mm Hg (≧140/≧90 mm) and urine protein collection ≧0.3 g/24 h) are enrolled. Exclusion criteria include HELLP syndrome (alanine aminotransferase >70 U/L, platelets <100×10³/mcl, evidence of hemolysis on blood smear), oliguria (<500 mL/24h) or evidence of acute renal failure (creatinine value >2.5 mg/L) or oligohydramnios (index <5 cm).

In addition to usual standard of care, patients are randomly assigned to receive ATryn® (BID, i.e., twice daily, bolus infusions or a 24-hours continuous infusion) or placebo, for an expected duration of approximately 7 to 15 days, until maternal/fetal deterioration and/or indication for both expectant management cessation and delivery. One of the ATryn® doses evaluated is a dose of 12000 units per day through continuous infusion. The primary endpoint is an increase in gestational age from randomization to delivery. Safety assessments comprise adverse events (AEs), serious AEs, and a priori complications of interest (e.g., death, seizures, cardiovascular events, bronchopulmonary dysplasia). Laboratory assays are used to monitor AT activity levels, coagulation, urine protein, and other biomarkers. Maternal and neonatal complication assessments will continue until discharge; neonatal follow-up will continue up to 6 months after delivery.

### Example 2

### Treatment of Severe Pre-Eclampsia with ATryn®

Severe pre-eclamptic patients between 24-36 weeks of gestation receive ATryn® (1500 U/day). ATryn® is given intravenously once daily for 7 consecutive days.

Therapeutic efficacy is evaluated by determining the gestosis index, which is composed of Proteinurea (gram protein/L), systolic blood pressure and diastolic blood pressure. Therapeutic efficacy is further evaluated by determining coagulation parameters (level of thrombin-antithrombin complexes, plasmin-plasmin inhibitor complex and D-dimer). In addition, biophysical characteristics of the newborns, such as heart rate and breathing movements, are evaluated, (See Kobayashi et al. Semin. Thromb. Hemost. 2003, 29: 645-652).

### Example 3

### Treatment of Pre-Eclampsia using High Doses of ATryn®.

Pre-eclamptic patients are treated either with high doses of ATryn® (3000 units) once daily for 5 days, or until delivery of the newborn, or patients are treated with doses of ATryn® sufficient to maintain at least 80% of baseline antithrombin activity.

A first endpoint for determining therapeutic efficacy is the prolongation of pregnancy defined as time (in days) from enrollment to delivery and the maternal bleeding at and after delivery. A secondary endpoint is the evaluation of the role of ATryn® in controlling hemostasis as determined by antithrombin activity and the levels of fibronectin (Fn), fibrinogen, D-dimer, uricemia, proteinuria 24h, reactive C protein, granulocyte elastase and endothelin (See Paternoster et al. Thromb Haemost 2004, 91: 283-289).

### Equivalents

The foregoing written specification is considered to be sufficient to enable one skilled in the art to practice the invention. The present invention is not to be limited in scope by examples provided, since the examples are intended as an illustration of certain aspects and embodiments of the invention. Other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method of treating pre-eclampsia in a subject, the method comprising:
administering to a subject having pre-eclampsia a therapeutically effective amount of antithrombin to treat the pre-eclampsia.

2. The method of claim 1, wherein the pre-eclampsia is severe pre-eclampsia.

3. The method of claim 1 or claim 2, wherein the subject is at less than 24 weeks of pregnancy.

4. The method of claim 1 or claim 2, wherein the subject is at between 24 weeks and 28 weeks of pregnancy.

5. The method of claim 1 or claim 2, wherein the subject is at between 28 weeks and 32 weeks of pregnancy.

6. The method of claim 1 or claim 2, wherein the subject is at between 28 weeks and 34 weeks of pregnancy.

7. The method of claim 1 or claim 2, wherein the subject is at over 34 weeks of pregnancy.

8. The method of any one of claims 1-7, wherein the antithrombin has a high mannose glycosylation pattern.

9. The method of any one of claims 1-8, wherein the antithrombin comprises GalNac (N-acetylgalactosamine).

10. The method of any one of claims 1-9, wherein the antithrombin has a fucose-GlcNAc glycosylation pattern.

11. The method of any one of claims 1-10, wherein the antithrombin is transgenically produced antithrombin.

12. The method of claim 11, wherein the antithrombin is transgenically produced in a goat.

13. The method of any one of claims 1-12, wherein the antithrombin is ATryn®.

14. The method of any one of claims 1-13, wherein the antithrombin is administered at a dose of 1500 units per day.

15. The method of any one of claims 1-13, wherein the antithrombin is administered at a dose of 3000 units per day.

16. The method of any one of claims 1-13, wherein the antithrombin is administered at a dose of 12,000 units per day.

17. The method of any one of claims 1-16, wherein the antithrombin is administered through continuous infusion.

18. The method of any one of claims 1-16, wherein the antithrombin is administered as a bolus.

19. The method of any one of claims 1-13, wherein the antithrombin is administered at a dose of 3500 units twice daily by bolus infusion.

20. The method of any one of claims 1-13, wherein the antithrombin is administered at a dose of 10,500 units daily by continuous infusion.

21. The method of any one of claims 1-13, wherein the antithrombin is administered at a dose of 12,000 units daily by continuous infusion.
